(19) United States
(12) Patent Application Publication
(10) Pub. No.: US 2024/0336733 A1
(43) Pub. Date: Oct. 10, 2024

(54) AMINATION PROCESS OF MICROMOLECULAR POLYOXYPROPYLENE ETHER

(71) Applicants: ZHEJIANG HUANGMA TECHNOLOGY CO., LTD., Shaoxing (CN); ZHEJIANG LUKEAN CHEMICAL CO., LTD., Shaoxing (CN); ZHEJIANG HUANGMA CHEMICAL NEW POLYMER MATERIAL CO., LTD, Shaoxing (CN); ZHEJIANG HUANGMA SHANGYI NEW MATERIAL CO., LTD., Shaoxing (CN)

(72) Inventors: Weisong Wang, Shaoxing (CN); Yifeng Jin, Shaoxing (CN); Jiang Yu, Shaoxing (CN); Minggui Song, Shaoxing (CN); Majishi Wang, Shaoxing (CN)

(21) Appl. No.: 18/628,980

(22) Filed: Apr. 8, 2024

(30) Foreign Application Priority Data
Apr. 10, 2023 (CN) 202310369374.X

(51) Int. Cl. C08G 65/333

(52) U.S. Cl. CPC C08G 65/33306

(57) ABSTRACT

The present disclosure relates to an amination production method of micromolecular polyoxypropylene ether, belonging to the technical field of preparation or chemical processing of organic compounds. An amination process of the micromolecular polyoxypropylene ether includes the following steps: (1) adding the micromolecular polyoxypropylene ether into a stirring type reaction kettle, introducing liquid ammonia and hydrogen, and implementing terminal hydroxyl amination on the micromolecular polyoxypropylene ether in presence of a catalyst I cat-1 at a lower temperature within a shorter reaction time, so as to obtain amine-terminated polyoxypropylene ether with an amination rate being about 70%-80%; and (2) introducing hydrogen, liquid ammonia, and the crude amine-terminated polyoxypropylene ether from the kettle type reaction liquid into a tubular reactor, and carrying out a hydroamination reaction in presence of a catalyst II cat-2 to obtain amine-terminated polyoxypropylene ether with an amination rate being about 98% or more.

### CROSS-REFERENCE TO RELATED APPLICATION

The present application claims priority from Chinese Patent Application No. 202310369374.X filed on Apr. 10, 2023, the contents of which are incorporated herein by reference in their entirety.

### TECHNICAL FIELD

The present disclosure relates to an amination production method of micromolecular polyoxypropylene ether, belonging to the technical field of preparation or chemical processing of organic compounds.

### BACKGROUND

Amine-terminated polyoxypropylene ether, also called polyether amine (PEA), is a kind of polymer whose main chain has a polyether structure and terminal active functional group is an amine group. Aminated polyether is formed by converting the hydroxyl groups of polyethylene glycol, polypropylene glycol, or ethylene glycol/propylene glycol copolymers into amine groups. By choosing different polyoxyalkyl structures, a series of properties such as reactivity, toughness, viscosity and hydrophilicity of the amino-terminated polyoxypropylene ether can be adjusted, while a terminal amino group provides the possibility for the amino-terminated polyoxypropylene ether to react with various compounds. The special molecular structure of the amino-terminated polyoxypropylene ether endows the amino-terminated polyoxypropylene ether with excellent comprehensive properties. The current commercialized amine-terminated polyoxypropylene ethers include monofunctional, difunctional, and trifunctional series of products with molecular weights ranging from 230 to 5,000. Such compounds can be widely applied to the fields of epoxy resin curing agent, wind energy blade curing agent, polyurethane polyurea elastomer, gasoline cleaning agent, water-based coating, textile finishing agent and epoxy toughening.

At present, the mainstream synthesis method for industrialization is the catalytic amination method, which has the advantages of being stable in product quality and better in compliance with environmental protection requirements when being used for synthesizing amino-terminated polyoxypropylene ether. The catalytic amination method involves an amination reaction between polyether and liquid ammonia under high temperature and pressure in a hydrogen atmosphere for production of the corresponding polyether amine. For polyethers with different structural distributions and molecular weights, the main reaction modes of a hydrogenation reaction include intermittent high-pressure reactor operation and continuous fixed bed operation.

Compared with the continuous fixed bed process, the intermittent high-pressure reactor operation is higher reaction temperature and pressure, and longer in reaction time. Furthermore, an intermittent high-pressure reactor promotes the mixing of reaction raw materials by stirring, but the stirring causes serious damage to a catalyst, which in turn affects the service life of the catalyst. For polyether polyols with an average molecular weight of 600 or less, if a continuous reaction is adopted, the space velocity is low, and the reaction temperature is high. Low space velocity is not conducive to the release of production capacity, and the longer the residence time of low molecular weight polyether in a tubular reactor, the more obvious the reaction cracking and polymerization phenomena. The catalyst surface is prone to carbon deposition, resulting in activity reduction or deactivation of the catalyst.

Based on this, the present disclosure proposes the following application: In view of the advantages of a kettle reaction and a tubular reaction, a two-step method is used to synthesize micromolecular amino-terminated polyoxypropylene ether. Through the reaction in a high-pressure reactor, 70% to 80% of the micromolecular polyoxypropylene ether is terminated within a short period of time, and then the organic amine impurities are removed through deamination and dehydration processes; and then termination is continued through a tubular reactor. Since the amination rate has been completed by 70%- 80% in the first step, the second step can increase the space velocity and reduce the contact time of a catalyst. The removal of organic amine impurities in the first step reduces the impact of carbon deposition on the catalyst activity in subsequent reactions.

### SUMMARY

In response to the above-mentioned defects in the processing of the existing amino-terminated micromolecular polyoxypropylene ether, the present application provides an amination process of micromolecular polyoxypropylene ether. To achieve the above objectives, the technical solution adopted in the present application is as follows: an amination process of micromolecular polyoxypropylene ether includes the following steps:

An amination process of micromolecular polyoxypropylene ether includes the following steps:

(1) adding the micromolecular polyoxypropylene ether into a stirring type reaction kettle, introducing liquid ammonia and hydrogen, and implementing terminal hydroxyl amination on the micromolecular polyoxypropylene ether in presence of a catalyst I cat-1 to obtain amine-terminated polyoxypropylene ether with an amination rate being about 70%- 80%, where the molar ratio of the liquid ammonia to the micromolecular polyoxypropylene ether is (2-20): 1, and after the addition of the micromolecular polyoxypropylene ether and the liquid ammonia, the hydrogen is introduced to allow the pressure in the reaction kettle to reach 1.0-2.0 Mpa;

(2) introducing hydrogen, liquid ammonia, and the crude amine-terminated polyoxypropylene ether from the kettle type reaction liquid into a tubular reactor, where the molar ratio of the liquid ammonia to the micromolecular polyoxypropylene ether is (2-20): 1, and after the addition of the liquid ammonia and the crude amine-terminated polyoxypropylene ether from the kettle type reaction liquid, the hydrogen is introduced to allow the pressure in the reaction kettle to reach 1.0-2.0 Mpa initially; and carrying out a hydroamination reaction in presence of a catalyst II cat-2 to obtain amine-terminated polyoxypropylene ether with an amination rate being about 98% or more.

In step (1), the catalyst I Cat-1 is a copolymer obtained by reduction roasting after a carrier MgAl204 is loaded with metal salts of Ni, Pt, and La; and in step (2), the catalyst II Cat-2 is composed of a carrier and metal salts loaded thereon, with the carrier selected from y-Al2O3, and the metal salts being Ni, Co, and Mo.

In step (1), the reaction temperature is 90-150° C., the absolute reaction pressure is 3.0-8.0MPa, and the reaction time is 2.0-5.0 h.

In step (2), the reaction temperature is 110-170° C., the absolute reaction pressure is 5.0-10.0MPa, and the space velocity is 0.1-0.2 g/h/g Cat.

In step (2), the addition molar ratio of the liquid ammonia to the micromolecular polyoxypropylene ether is (5-10):1, and the addition molar ratio of the hydrogen to the micromolecular polyoxypropylene ether is (0.1-2):1.

In step (1), the catalyst I is a carrier MgAl204 loaded with three metal salts, i.c., Ni (NO₃)₂, Pt(NO₃)₂, and La(NO₃)₃.

In step (1), reduction roasting is performed at 350-500° C. during the preparation of the catalyst I.

In step (1), the metal contents in the catalyst I Cat-1 are 0.5-7.0% of Ni, 0.1-0.5% of Pt, 0.4-2.5% of La, 14.0-18.0% of Mg, and 26.0-36.0% of Al.

In step (2), the metal salts in the catalyst II are Ni (NO₃)₂·6H₂O, Co (NO₃)₂·6H₂O, and Mo(NO₃)₃·5H2O.

In step (2), the metal contents in the catalyst II Cat-2 are 0.5-5.0% of Ni, 0.5-3.0% of Co, 0.2-2.0% of Mo, and 32.0-42.0% of Al.

The structure of the micromolecular polyoxypropylene ether is formed by polymerizing propylene glycol with ethylene oxide and/or propylene oxide, and the molecular weight thereof is 50-600.

The present disclosure is applied to micromolecular amino-terminated polyoxypropylene ether. The polyether is fully contacted with the catalyst through the kettle reaction in the first step, and most of hydroxyl groups are aminated at a lower temperature within a shorter reaction time; the polyether is fully contacted with the catalyst through the reaction in the first step, so that the generation of by-products is reduced, and the space velocity of the subsequent continuous reactions can be effectively improved; and the continuous reaction in the second step not only effectively makes up for the defect of the low amination rate of the kettle reaction, but also prolongs the service life of the kettle catalyst and reduces the process difficulty of the kettle reaction.

### DETAILED DESCRIPTION OF THE EMBODIMENTS

Preparation method of micromolecular polyoxypropylene ether:
3 mol (228 g) of propylene glycol was added into a reaction kettle, 3.0 g of KOH was then added, and the obtained mixture was heated up to 100-110° C. for dehydration for 30 min. The product was heated again and introduced with 9 mol (540 g) of propylene oxide slowly. During the introduction of propylene oxide, the temperature was kept within 115-125° C. After the addition of propylene oxide was finished, the reaction was continued for 30 min before discharging. The initial product polyether was absorbed by an adsorbent, and then micromolecular polyether with a molecular weight of 240 was obtained.

### Example 1. Preparation of Catalyst CAT-1

A catalyst was prepared according to an impregnation method. Metals Pt, Ni, and La in amounts accounting for 0.25%, 3.5%, and 1.2% of the total weight of the catalyst were prepared into an aqueous solution with a certain concentration of three metal salts, i.c., Ni (NO₃)₂, Pt(NO₃)₂, and La (NO₃)₃. Impregnation was performed for 5 h at 70° C. until MgAl₂O₄ particles accounting for 95% of the total amount of the catalyst were formed on a spherical carrier. After being dried, the catalyst was subjected to reduction roasting at 420° C., so that a catalyst CAT-1 was obtained.

### Example 2. Preparation of Catalyst CAT-2

Metal salts, i.c., Ni (NO3)₂·6H2O, Co (NO₃)₂·6H₂O and Mo (NO₃)₃·5H2O were dissolved into a solution at a certain temperature according to a certain proportion: the metal contents were 2.5% of Ni, 2.0% of Co, and 1.0% of Mo. 300g of a γ-Al₂O₃ carrier was impregnated in batches for 50 min, and then the water was evaporated to dryness under the conditions that the water temperature was 70° C. and the vacuum degree was −0.080 to −0.095 MPa. After that, the evaporated processed material was placed in a vacuum oven at 120° C. for dehydration for about 3h to remove free water and crystalline water. After drying, the product was placed in a muffle furnace for roasting. The roasting was performed at 450° C. for 3h, the product was then put into a tubular high-temperature furnace for reduction under hydrogen atmosphere, the reduction was performed at the temperature of about 420° C. for 3h, and finally, a catalyst II (Cat-2) was obtained, where the metal contents in the γ-Al₂O₃ were 2.5% of Ni, 2.0% of Co, and 1.0% of Mo.

### Example 3. Catalyst use in Kettle Reaction

The catalysts of Examples 1 and 2 were selected respectively, and micromolecular polyoxypropylene ether was prepared by a kettle reactor. The amination reaction temperature was 120° C., the absolute reaction pressure was 7.0 MPa, and the molar ratio of liquid ammonia to the micromolecular polyoxypropylene ether was 10:1, that was, 340 g of the liquid ammonia and 460 g of the polyoxypropylene ether 230 were available in the reaction. After the addition of the micromolecular polyoxypropylene ether and the liquid ammonia was finished, hydrogen was introduced to make the reactor pressure reach 2.0 MPa. The reaction lasted for 4 h. The adaptability of the catalysts loaded on two kinds of carriers to the kettle reaction was investigated.

TABLE 1
Performance comparison of catalysts loaded on different carriers

| | Reaction | Service | Average | Primary | Average |
|---|---|---|---|---|---|
| Type of | pressure | life | amination | amine | crushing rate |
| catalyst | (MPa) | (times) | rate % | selectivity % | (%) |
| Catalyst of | 7.0 | 24 | 97.7 | 97.5 | 0.26 |
| Example 2 | | | | | |
| Catalyst of | 7.0 | 45 | 98.9 | 99.0 | 0.11 |
| Example 1 | | | | | |

Table 1 shows that the MgAl₂O₄ carrier is loaded with metal elements such as Pt, Ni, and La in the kettle reaction, the low crushing rate and the long service life indicate that the catalyst is very suitable for the kettle amination reaction, and the average amination rate and the selectivity indicate that the catalyst (CAT-1) has higher activity.

### Example 4

A catalyst CAT-1 was added to a high-pressure reactor, micromolecular polyoxypropylene ether 230 (460 g, 2 mol) was added, liquid ammonia (170 g, 10 mol) was then added, hydrogen was introduced until the initial pressure of the high-pressure reactor was 1.5 MPa, and the reaction temperature and the reaction pressure were respectively controller to be was 130° C. and 7.0 MPa. Under these reaction conditions, the effects of different reaction time on the amination rate and selectivity were investigated. The calculation method was: amination rate=total amine value/hydroxyl value of micromolecular polyoxypropylene ether*100%; amination selectivity=primary amine value/total amine value*100%. The determination method for total amine value and tertiary amine value was HMJC/ZD (J)-027-2023. The determination method for primary amine content was HMJC/ZD (J)-037-2023. The determination method for hydroxyl value was GB/T 7383.

TABLE 2
Test results of kettle reactor

| Reaction time (h) | Amination rate % | Primary amine selectivity % |
|---|---|---|
| 1 | 42.0 | 100 |
| 2 | 72.0 | 99.7 |
| 3 | 83.0 | 99.6 |
| 10 | 89.0 | 99.5 |
| 12 | 94.5 | 99.4 |
| 15 | 97.0 | 99.4 |

As can be seen from Table 2, the amination rate (less than 80%) of the kettle reaction has a fast reaction rate, which can usually be achieved within 3-5h. However, to further increase the amination rate, it requires several times longer reaction time, which is not conducive to the service life of the catalyst and the increase in production capacity.

### Example 5

A catalyst CAT-2 was loaded into a tubular reactor for reaction, and polyoxypropylene ether (230) was selected to react with liquid ammonia and hydrogen for amination at a reaction temperature of 150° C. and a reaction pressure of 8.0 MPa. The molar ratio of the liquid ammonia to the micromolecular polyoxypropylene ether was 7:1, and the molar ratio of the hydrogen to the micromolecular polyoxypropylene ether was 1:1. The amination rate and selectivity of different treated micromolecular polyoxypropylene ethers in the reaction system were compared.

TABLE 3
Test results of tubular reactor

| | | Feed | | | |
|---|---|---|---|---|---|
| | Organic | space | | | |
| | amine | velocity | | | |
| Polyoxypropylene | impurities | g/h/g/ | Amination | | Lifetime |
| ether | % | Cat | rate % | Selectivity | (h) |
| Unaminated | 0 | 0.05 | 85.5 | 99.8 | / |
| Aminated by 50% | 0 | 0.1 | 95.8 | 99.6 | / |
| Aminated by 70% | 1.0 | 0.1 | 99.2 | 99.2 | 2400 |
| Aminated by 70% | 0 | 0.15 | 99.8 | 99.8 | 3100 |
| Aminated by 80% | 1.5 | 0.1 | 99.0 | 93.2 | / |
| Aminated by 80% | 0 | 0.15 | 99.8 | 99.5 | / |

It can be seen from Example 5 that the intermediate amination rate of the product subjected to amination in the reaction kettle of the first step is 70-80%, which can greatly improve the space velocity; and the organic amine impurities have a great influence on the selectivity of the product, and further evidence has shown that the impurities have a significant impact on the lifetime of the catalyst CAT-2.

The present disclosure has been described in detail above in conjunction with specific embodiments and exemplary examples, but these descriptions should not be construed as limiting the present disclosure. Those skilled in the art understand that without departing from the spirit and scope of the present disclosure, various equivalent replacements, modifications or improvements can be made to the technical solutions and embodiments of the present disclosure, all of which fall within the scope of the present disclosure. The protection scope of the present disclosure shall be subject to the appended claims.

What is claimed is:

1. An amination process of micromolecular polyoxypropylene ether, comprising the following steps:
(1) adding the micromolecular polyoxypropylene ether into a stirring type reaction kettle, introducing liquid ammonia and hydrogen, and implementing terminal hydroxyl amination on the micromolecular polyoxypropylene ether in presence of a catalyst I cat-1 to obtain amine-terminated polyoxypropylene ether with an amination rate being about 70%- 80%, wherein the molar ratio of the liquid ammonia to the micromolecular polyoxypropylene ether is (2-20): 1, and after the addition of the micromolecular polyoxypropylene ether and the liquid ammonia, the hydrogen is introduced to allow the pressure in the reaction kettle to reach 1.0-2.0 Mpa;
(2) introducing hydrogen, liquid ammonia, and the crude amine-terminated polyoxypropylene ether from the kettle type reaction liquid into a tubular reactor, wherein the molar ratio of the liquid ammonia to the micromolecular polyoxypropylene ether is (2-20): 1, and after the addition of the liquid ammonia and the crude amine-terminated polyoxypropylene ether from the kettle type reaction liquid, the hydrogen is introduced to allow the pressure in the reaction kettle to reach 1.0-2.0 Mpa initially; and carrying out a hydroamination reaction in presence of a catalyst II cat-2 to obtain amine-terminated polyoxypropylene ether with an amination rate being about 98% or more;
in step (1), the catalyst I Cat-1 being a copolymer obtained by reduction roasting after a carrier MgAl₂O₄ is loaded with metal salts of Ni, Pt, and La; and in step (2), the catalyst II Cat-2 being composed of a carrier and metal salts loaded thereon, with the carrier selected from γ-Al₂O₃, and the metal salts being Ni, Co, and Mo.

2. The amination process of the micromolecular polyoxypropylene ether according to claim 1, wherein in step (1), the reaction temperature is 90-150° C., the absolute reaction pressure is 3.0-8.0 MPa, and the reaction time is 2.0-5.0 h.

3. The amination process of the micromolecular polyoxypropylene ether according to claim 1, wherein in step (2), the reaction temperature is 110-170° C., the absolute reaction pressure is 5.0-10.0 MPa, and the space velocity is 0.1-0.2 g/h/g Cat.

4. The amination process of the micromolecular polyoxypropylene ether according to claim 1, wherein in step (2), the addition molar ratio of the liquid ammonia to the micromolecular polyoxypropylene ether is (5-10):1, and the addition molar ratio of the hydrogen to the micromolecular polyoxypropylene ether is (0.1-2):1.

5. The amination process of the micromolecular polyoxypropylene ether according to claim 1, wherein in step (1), the catalyst I is a carrier MgAl₂O₄ loaded with three metal salts, i.e., Ni(NO₃)₂, Pt(NO₃)₂, and La (NO₃)₃.

6. The amination process of the micromolecular polyoxypropylene ether according to claim 5, wherein in step (1), reduction roasting is performed at 350-500° C. during the preparation of the catalyst I.

7. The amination process of the micromolecular polyoxypropylene ether according to claim 5, wherein in step (1), the metal contents in the catalyst I Cat-1 are 0.5-7.0% of Ni, 0.1-0.5% of Pt, 0.4-2.5% of La, 14.0-18.0% of Mg, and 26.0-36.0% of Al.

8. The amination process of the micromolecular polyoxypropylene ether according to claim 1, wherein in step (2), the metal salts in the catalyst II are Ni (NO₃)₂·6H2O, Co (NO₃)₂·6H2O, and Mo(NO₃)₃·5H2O.

9. The amination process of the micromolecular polyoxypropylene ether according to claim 8, wherein in step (2), the metal contents in the catalyst II Cat-2 are 0.5-5.0% of Ni, 0.5-3.0% of Co, 0.2-2.0% of Mo, and 32.0-42.0% of Al.

10. The amination process of the micromolecular polyoxypropylene ether according to claim 1, wherein the structure of the micromolecular polyoxypropylene ether is formed by polymerizing propylene glycol with cthylene oxide and/or propylene oxide, and the molecular weight thereof is 50-600.
